(19) United States
(12) Patent Application Publication
(10) Pub. No.: US 2022/0325311 A1
(43) Pub. Date: Oct. 13, 2022

(54) Sugarless peptide cultivated enzyme

(71) Applicant: Jui-Hung Chen, Changhua (TW)

(72) Inventor: Jui-Hung Chen, Changhua (TW)

(21) Appl. No.: 17/647,983

(22) Filed: Jan. 14, 2022

(30) Foreign Application Priority Data
Apr. 8, 2021 (TW) 110112779

(51) Int. Cl. C12P 21/00; A23L 19/00

(52) U.S. Cl. CPC C12P 21/00; A23L 19/09; A23V 2002/00

(57) ABSTRACT

A process of preparing sugarless peptide cultivated enzyme is provided in which surfaces of vegetable and fruit are cleaned and sterilized by hyperbaric oxygen, the vegetable and fruit with flesh and skin are smashed to a vegetable and fruit paste having dregs, the dregs is separated from the vegetable and fruit paste which is then soaked to form a raw vegetable and fruit solution which is poured into a fermentation tank including a catalase peptide ceramic layer having graphene, the raw vegetable and fruit solution cyclically flows through the catalase peptide ceramic layer for 1.5 months to 3 months of fermentation and cultivation period, and the cultivated vegetable and fruit solution is filtered to obtain a liquid sugarless vegetable and fruit enzyme product.

### FIELD OF THE INVENTION

The invention relates to health food, and more particularly to a process of preparing a sugarless cultivated enzyme product.

### BACKGROUND OF THE INVENTION

Enzyme is a type of protein that acts as a catalyst in human body. Deficiency of enzyme in human body would affect the metabolic and functional activities, thus preventing the human body from maintaining normal function.

The common vegetable and fruit enzyme products are generally produced through typical fermentation process, in which the peeled vegetable and fruit are put in a container, and mixed with a fermentative strain. The fermentative strain is generally a laboratory cultured non-genetic strain in which, the yeast or probiotics added with granulated sugar, is fermented in a sealed container for more than 3 months to produce an enzyme product.

However, in the typical process of vegetable and fruit enzyme, the vegetable, fruit flesh, yeast or probiotics and granulated sugar need to be fermented for a long time, as the yeast or probiotics is intolerant of high temperature. The addition of granulated sugar is required to preserves the yeast or probiotics, while the fruit juice is released under the osmotic pressure. The vegetable and fruit enzyme products made with vegetables and fruits tend to have residual sugar content, and cannot be used by patients suffering from metabolic syndrome or diabetes. Long-term consumption of vegetable and fruit enzyme products may lead to higher risks of fatty liver, cancer, obesity, etc. Moreover, the vegetable and fruit enzyme products have low activity, generally about 8×10⁵ Unite per 100 ml, so that the enzyme products must be sterilized by high temperature, strong base or strong acid, or mixed with sugar for preservation, which is unhealthy to human body.

### SUMMARY OF THE INVENTION

Therefore, in order to solve the problems of producing enzyme product with residual sugar content in the general vegetable and fruit enzyme fermentation process and low activity of the produced enzyme product, the inventor of the invention developed a process of sugarless peptide cultivated enzyme.

The invention uses a natural genetic strain in the vegetable and fruit fermentation process, the natural genetic strain can inhibit the growth of harmful infectious microbes, so that the quality of the vegetable and fruit enzyme product of the invention could be easily maintained, and the high activity and high antioxidant capacity of enzyme product could be enhanced. The “activity” refers to the superoxide dismutase (SOD), the “high antioxidant capacity” refers to the Oxygen Radical Absorbance Capacity (ORAC); in other words, the SOD of the vegetable and fruit enzyme product of the invention is 3.21×10⁶ Unite to 4.18×10⁸ Unite per 100 ml (g), and the ORAC of the vegetable and fruit enzyme product of the invention is 2.08×10⁵ μM TE per 100 ml (g); additionally, this process is free of granulated sugar or sugar substitute, so there is not sugar content produced, so that the produced vegetable and fruit enzyme product produced is suitable for the patients suffering from metabolic syndrome or diabetes.

Moreover, the invention uses cyclically flowing raw liquor of vegetable and fruit in a fermentation tank, which automatically leads the aerial oxygen into the fruit juice enzyme liquid, as in the automatic stirring method. High purity oxygen can be driven into the fermentation tank as needed, and the raw liquor of vegetable and fruit flows through the catalase peptide ceramic layer repeatedly, so that the raw liquor of vegetable and fruit in the fermentation tank is ripened into a cultivation liquid earlier. High content SOD is cultivated in a short period, and the growth of infectious microbes can be inhibited. The time cost of enzyme process of the invention is shortened. Therefore, the primary object of the invention is to provide a process of sugarless peptide cultivated enzyme free of granulated sugar or other sugar substitutes which accelerates the vegetable and fruit enzyme product yield; another object of the invention is to provide a process of sugarless peptide cultivated enzyme which uses natural genetic strains and can enhance the activity of vegetable and fruit enzyme products.

To attain said objects, the invention uses the following technical means: a process for preparing liquid sugarless peptide cultivated enzyme, including a vegetable and fruit cleaning step, the surface of vegetable and fruit is preliminarily cleaned; a hyperbaric oxygen sterilization step, the surface of vegetable and fruit is sterilized, but the natural genetic strain still adheres to the vegetable and fruit surface; a vegetable and fruit grinding step, the vegetable and fruit with flesh and skin are smashed to a vegetable and fruit paste with dregs; a juice and dregs separation and soaking step for raw liquor of vegetable and fruit, the vegetable and fruit dregs are bagged and soaked in the raw liquor of vegetable and fruit to form a raw liquor of vegetable and fruit; a step of circular stirring of raw liquor of vegetable and fruit in the fermentation tank, the raw liquor of vegetable and fruit is placed in the fermentation tank with a catalase peptide ceramic layer that contains graphene, a part of the raw liquor of vegetable and fruit is sprayed from a high position to a low position to impact the surface of the raw liquor of vegetable and fruit, as in the automatic stirring method, so as to automatically lead the aerial oxygen into the raw liquor of vegetable and fruit; a step of accelerating the ripening of raw liquor of vegetable and fruit, the raw liquor of vegetable and fruit flows through the catalase peptide ceramic layer cyclically, in the 1.5 months to 3 months long fermentation cultivation period, the raw liquor of vegetable and fruit can ripen into a cultivation liquid rapidly after molecular nano-thinning; a vegetable and fruit residue filtering step, the vegetable and fruit cultivation liquid with dregs is filtered; and a cultivation liquid bottling step, the filtered cultivation liquid is bottled as a sugar free liquid vegetable and fruit enzyme product.

In addition, the process for preparing solid sugarless peptide cultivated enzyme of the invention uses a cold drying and freezing pulverization step to replace said cultivation liquid bottling step, the cultivation liquid is dried at low temperature before packaging in the cold drying and freezing pulverization step, it becomes a solid vegetable and fruit enzyme product free of sugar when it is reheated.

In said step of cyclically stirring the raw liquor of vegetable and fruit in the fermentation tank, high purity oxygen can be driven in the fermentation tank to accelerate the ripening of the raw liquor of vegetable and fruit in the fermentation tank into the cultivation liquid, cultivate a high content of SOD in a short period, and inhibit the growth of infectious microbes, the duration of overall process of the invention can be halved, meaning the overall product completion period is 0.75 to 1.5 months.

The SOD of said liquid vegetable and fruit enzyme product can be 3.21×10⁶ Unite to 3.17×10⁷ Unite per 100 ml.

Said vegetable and fruit are set as jaboticaba, pineapple, Noni fruit, curcuma, orange, apple, green plum, Indian gooseberry or their combinations, and the total peptide content in the product can be 437.92 mg/100 mL(g).

Said solid vegetable and fruit enzyme product is pastille, powder or capsule, and its activity is 2.68×10⁸ Unite to 4.18×10⁸ Unite per 100 g.

The above and other objects, features and advantages of the invention will become apparent from the following detailed description taken with the accompanying drawings

### BRIEF DESCRIPTION OF THE DRAWINGS

FIG. 1 is a flow chart of a process of preparing liquid vegetable and fruit enzyme product of a first preferred embodiment of the invention;

FIG. 2 is a flow chart of a process of preparing solid vegetable and fruit enzyme product of a second preferred embodiment of the invention;

FIG. 3 is a side elevation in part section of the fermentation tank of the invention;

FIG. 4 is a perspective view of the catalase peptide ceramic layer of the invention; and

FIG. 5 is a table showing the invention being capable of enhancing the activity (SOD) and the total amount of peptide in the enzyme products.

### DETAILED DESCRIPTION OF THE INVENTION

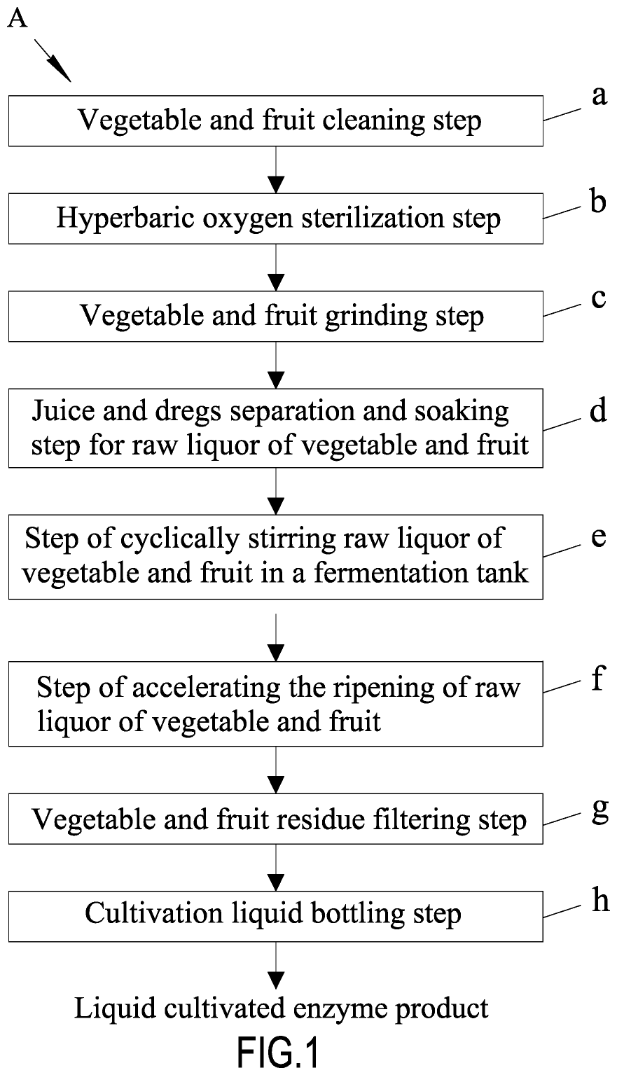

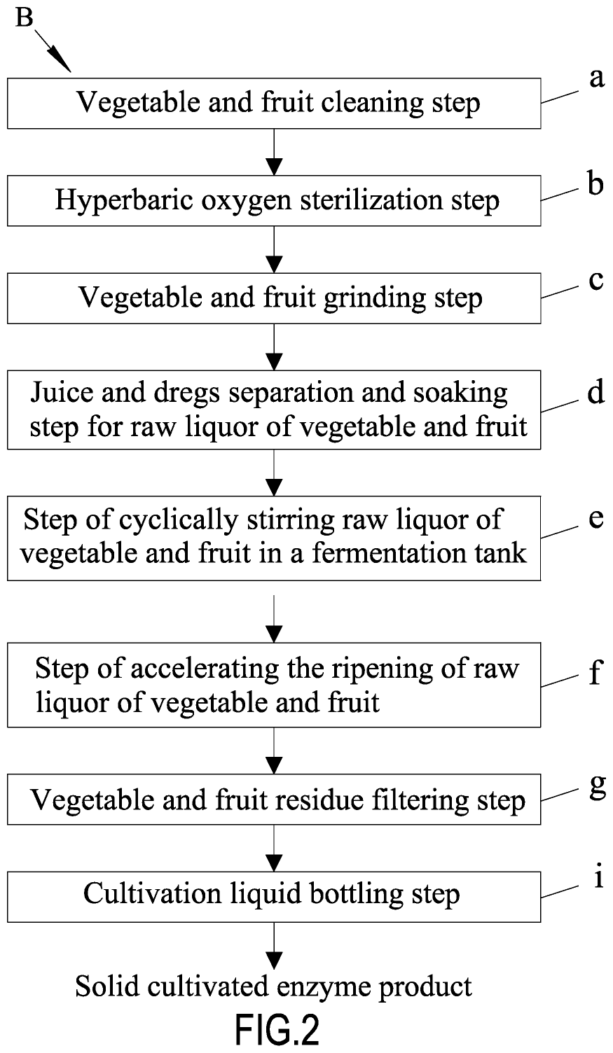

Referring to FIG. 1 and FIG. 2, the invention is a process A for preparing liquid sugarless peptide cultivated enzyme and a process B for preparing solid sugarless peptide cultivated enzyme respectively. The sugar content in the fruit is used as nutrient, the cultivated genetic strain is used for secondary cultivation, and a fermentation tank with catalase peptide ceramic layer is used for 10⁻¹² high frequency oscillation to generate resonance and infiltration, the molecules of vegetable and fruit cultivation liquid are nano-refined rapidly, the SOD activity and the total amount of peptide in the enzyme product is enhanced in a very short period of time, so as to inhibit the growth of harmful infectious microbes, detailed below:

Specifically, FIG. 1 illustrates a process A for preparing liquid sugarless peptide cultivated enzyme of a first preferred embodiment of the invention, including a vegetable and fruit cleaning step a, the surface of vegetable and fruit is preliminarily cleaned; a hyperbaric oxygen sterilization step b, the surface of vegetable and fruit is sterilized, but there are still natural genetic strains adhering to the surface of vegetable and fruit; a vegetable and fruit grinding step c, the vegetable and fruit with natural genetic strains adhering to their surface are smashed with flesh and skin into a vegetable and fruit paste with dregs; a juice and dregs separation and soaking step d for raw liquor of vegetable and fruit, the vegetable and fruit dregs are bagged by a filter screen and soaked in the raw liquor of vegetable and fruit to become a raw liquor of vegetable and fruit 3; particularly, said vegetable and fruit refer to jaboticaba, pineapple, Noni fruit, curcuma, orange, apple, green plum, Indian gooseberry or their combinations, additionally, the natural genetic strain is active and can withstand a high temperature above 125° C., so it is unnecessary to use sugar to enhance the activity.

Further, there is a step e of pouring the raw liquor of vegetable and fruit 3 into the fermentation tank 1 until a space of the fermentation tank 1 under the catalase peptide ceramic layer 2 is filled with a predetermined quantity of the raw liquor of vegetable and fruit 3; and cyclically stirring the raw liquor of vegetable and fruit 3 in the fermentation tank 1, a quantity of the raw liquor of vegetable and fruit 3 is sprayed from a high position to a low position to impact the surface 31 of the raw liquor of vegetable and fruit 3, like automatic stirring, so as to automatically lead the aerial oxygen in the raw liquor of vegetable and fruit 3, it is approximate to the principle of boom circulating water, unicell multiplication can be inhibited; as the catalase peptide ceramic layer 2 has 10⁻¹² high frequency oscillation and 30μ wavelength radiation, the raw liquor of vegetable and fruit 3 generates multicellular resonance, the molecules are nano-refined to form activity (SOD). Particularly, high purity oxygen can be driven in the fermentation tank 1 in this step, so as to accelerate the ripening of the raw liquor of vegetable and fruit 3 in the fermentation tank 1 into a cultivation liquid, cultivate a high content of SOD in a short period and inhibit the growth of infectious microbes, the duration of overall process of the invention can be approximately halved, meanwhile the quality of product is enhanced, meaning the overall product completion period is 0.75 to 1.5 months; the UV or O₃ is used for sterilization in the automatic stirring process if necessary.

Further, there is a step f of accelerating the ripening of raw liquor of vegetable and fruit, the raw liquor of vegetable and fruit 3 flows through the catalase peptide ceramic layer 2 cyclically, in the 1.5 months to 3 months long fermentation cultivation period, the raw liquor of vegetable and fruit 3 can ripen into the cultivation liquid rapidly as the molecules are nano-refined, the fermentation period is shorter than typical process.

The process A of the invention has a vegetable and fruit residue filtering step g, the vegetable and fruit cultivation liquid with dregs is filtered; and a cultivation liquid bottling step h, the cultivation liquid is bottled to become a liquid vegetable and fruit enzyme product free of sugar.

Specifically, FIG. 2 illustrates a process B for preparing solid sugarless peptide cultivated enzyme of a second preferred embodiment of the invention, the difference from said process A for preparing liquid sugarless peptide cultivated enzyme is that the process B is free of the cultivation liquid bottling step h, the cultivation liquid bottling step h of the process A is replaced by a low temperature and freezing pulverization drying step i, wherein the cold drying and freezing pulverization step i dries the cultivation liquid at low temperature to solid state, which is reheated and packaged to become a solid vegetable and fruit enzyme product free of sugar, and the solid vegetable and fruit enzyme product can be pastille, powder or capsule.

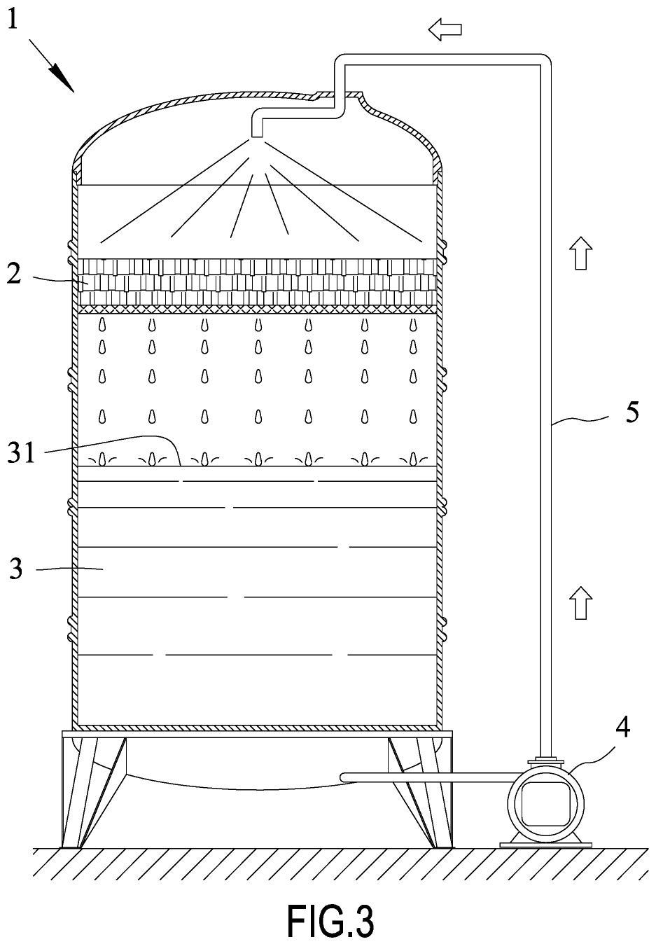

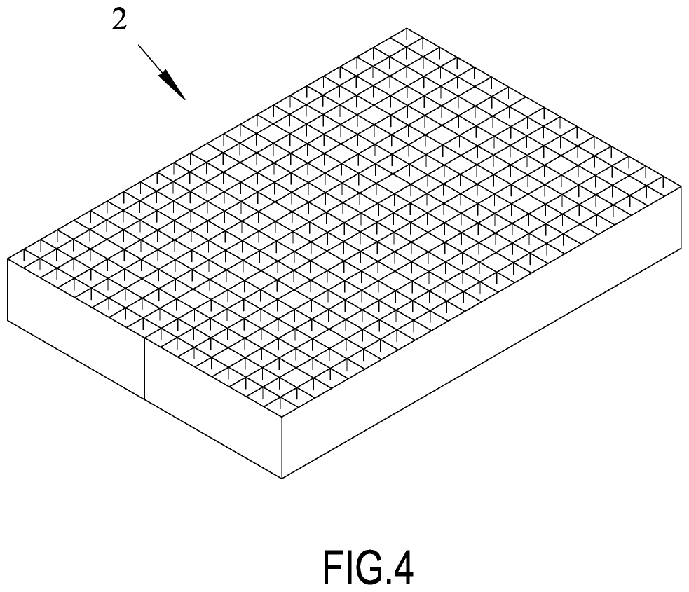

Referring to FIGS. 3 to 5 in conjunction with FIGS. 1 and 2, they show the operation mode of the fermentation tank 1 used in said process A for preparing liquid sugarless peptide cultivated enzyme and process B for preparing solid sugarless peptide cultivated enzyme, wherein the raw liquor of vegetable and fruit 3 containing natural genetic strains is stored, when a pump 4 arranged outside the fermentation tank 1 is actuated, the pump 4 pumps the raw liquor of vegetable and fruit 3 from the bottom of the fermentation tank 1, which is sprinkled on the catalase peptide ceramic layer 2 in the fermentation tank 1 from top of the fermentation tank 1 through an external pipeline 5, the raw liquor of vegetable and fruit 3 falls and hits the surface 31 of static raw liquor of vegetable and fruit 3 in the tank 1, like leading the aerial oxygen in the raw liquor of vegetable and fruit 3, and then the raw liquor of vegetable and fruit infiltrates into the catalase peptide ceramic layer 2 to resonate oscillation and accelerate the curing of the raw liquor of vegetable and fruit.

Therefore, a process A for preparing liquid sugarless peptide cultivated enzyme and a process B for preparing solid sugarless peptide cultivated enzyme of the invention use natural genetic strains, the growth of harmful infectious microbes can be inhibited in the fermentation process, the pH of the high SOD sugarless vegetable and fruit enzyme product of the invention is lower than 3.5, this condition is unfavorable for the multiplication of infectious microbes, so the processes of the invention do not need high temperature, strong base or strong acid for sterilization at all, or use sugar for preservation, and the sugarless vegetable and fruit enzyme product of the invention can withstand strong acid up to pH0.9, it will not be damaged by the gastric acid of human body, so it is easy to maintain the quality of sugarless enzyme product of the invention, the user's health can be enhanced.

As the process of the invention is free of granulated sugar or other sugar substitutes, it will not produce vegetable and fruit enzyme products with sugar content, meaning the produced vegetable and fruit enzyme product is good for the patients suffering from metabolic syndrome or diabetes to drink or eat.

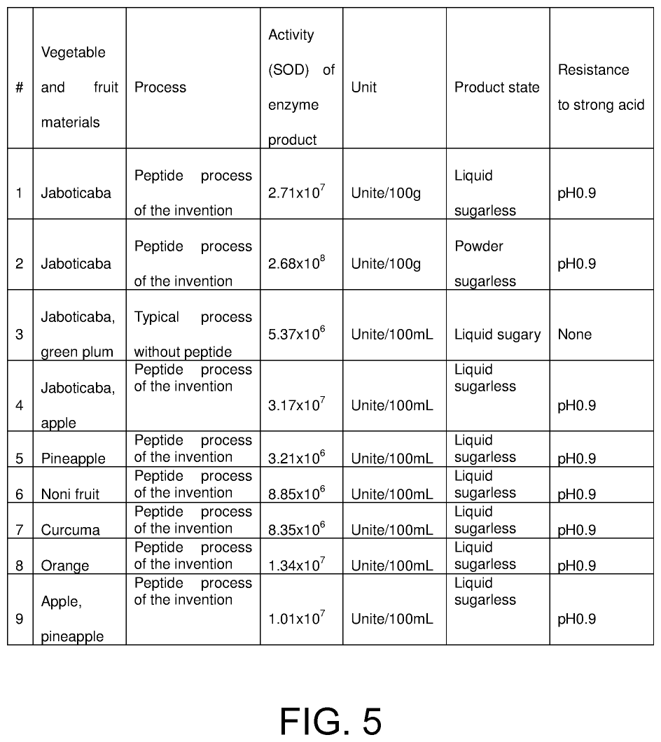

The invention can enhance the activity (SOD) and the total amount of peptide in the enzyme products, as shown in the attached documentary evidence of this project of FIG. 5.

The activity (SOD) of liquid enzyme product of the invention is 3.21×10⁶ Unite to 3.17×10⁷ Unite per 100 ml, and the activity (SOD) of solid enzyme product of the invention is 2.68×10⁸ Unite to 4.18×10⁸ Unite per 100 g; taking jaboticaba as an example, in comparison to the sugary enzyme product of typical process, the sugarless cultivated enzyme product of the invention can enhance the activity (SOD) of vegetable and fruit enzyme product; meanwhile the total peptide content in the vegetable and fruit enzyme product of the invention can be 437.92 mg/100 mL (g), and the ORAC can be 2.08×10⁵ μMTE per 100 ml (g).

Further, the invention cyclically flows raw liquor of vegetable and fruit in the fermentation tank 1, like automatically leading aerial oxygen in the fruit juice enzyme liquid by automatic stirring, and high purity oxygen can be driven in the fermentation tank 1 as required, the raw liquor of vegetable and fruit 3 flows through the catalase peptide ceramic layer 2 repeatedly, so as to accelerate the ripening of the raw liquor of vegetable and fruit 3 in the fermentation tank 1 into a cultivation liquid, cultivate a high content of SOD in a short period and inhibit the growth of infectious microbes, the time cost of enzyme process of the invention can be reduced and the quality of product can be enhanced.

While the invention has been described in terms of preferred embodiments, those skilled in the art will recognize that the invention can be practiced with modifications within the spirit and scope of the appended claims.

What is claimed is:

1. A process of preparing sugarless peptide cultivated enzyme, comprising the steps of:
(a) cleaning both vegetable and fruit;
(b) performing hyperbaric oxygen sterilization on surfaces of both the vegetable and fruit with natural genetic strains adhered to the surfaces of both the vegetable and fruit;
(c) grinding both the vegetable and fruit until flesh and skin of both the vegetable and fruit are smashed to a vegetable and fruit paste having dregs;
(d) separating the dregs from the vegetable and fruit paste to obtain a vegetable and fruit mixture;
(e) soaking the vegetable and fruit mixture until a raw vegetable and fruit solution is formed;
(f) pouring the raw vegetable and fruit solution into a fermentation tank including an internal, horizontal catalase peptide ceramic layer having graphene until a space of the fermentation tank under the catalase peptide ceramic layer is filled with a predetermined quantity of the raw vegetable and fruit solution;
(g) cyclically stirring the raw vegetable and fruit solution by pumping the raw vegetable and fruit solution and spraying same from a top of the fermentation tank on and through the catalase peptide ceramic layer to mix with the raw vegetable and fruit solution and introducing oxygen of the air into the raw vegetable and fruit solution;
(h) performing step (g) for 1.5 months to 3 months to ripen the raw vegetable and fruit solution until a cultivated vegetable and fruit solution is formed; and
(i) filtering the cultivated vegetable and fruit solution to obtain a liquid vegetable and fruit enzyme product free of sugar.

2. The process of claim 1, wherein step (g) comprises the sub-steps of introducing both pure oxygen and O₃ into the fermentation tank.

3. The process of claim 1, wherein activity of the liquid vegetable and fruit enzyme product is from 3.21×10⁶ Unite to 3.17×10⁷ Unite (SOD) per 100 ml (g).

4. The process of claim 1, wherein both the vegetable and fruit are selected from the group consisting of jaboticaba, pineapple, Noni fruit, curcuma, orange, apple, green plum, Indian gooseberry and any combinations thereof, and a total peptide content in the liquid vegetable and fruit enzyme product is 437.92 mg/100 mL.

5. A process of preparing sugarless peptide cultivated enzyme, comprising the steps of:
(a) cleaning both vegetable and fruit;
(b) performing hyperbaric oxygen sterilization on surfaces of both the vegetable and fruit with natural genetic strains adhered to the surfaces of both the vegetable and fruit;
(c) grinding both the vegetable and fruit until flesh and skin of both the vegetable and fruit are smashed to a vegetable and fruit paste having dregs;
(d) separating the dregs from the vegetable and fruit paste to obtain a vegetable and fruit mixture;
(e) soaking the vegetable and fruit mixture until a raw vegetable and fruit solution is formed;
(f) pouring the raw vegetable and fruit solution into a fermentation tank including an internal, horizontal catalase peptide ceramic layer having graphene until a space of the fermentation tank under the catalase peptide ceramic layer is filled with a predetermined quantity of the raw vegetable and fruit solution;
(g) cyclically stirring the raw vegetable and fruit solution by pumping the raw vegetable and fruit solution and spraying same from a top of the fermentation tank on and through the catalase peptide ceramic layer to mix with the raw vegetable and fruit solution and introducing oxygen of the air into the raw vegetable and fruit solution;
(h) performing step (g) for 1.5 months to 3 months to ripen the raw vegetable and fruit solution until a cultivated vegetable and fruit solution is formed; and
(i) drying and freezing the cultivated vegetable and fruit solution to obtain a solid vegetable and fruit enzyme product free of sugar.

6. The process of claim 5, wherein step (g) comprises the sub-steps of introducing both pure oxygen and O₃ into the fermentation tank.

7. The process of claim 5, wherein activity of the solid vegetable and fruit enzyme product is 2.68×10⁸ Unite to 4.18×10⁸ Unite per 100 g, and the ORAC is 2.08×10⁵ μM TE per 100 g.

8. The process of claim 5, wherein both the vegetable and fruit are selected from the group consisting of jaboticaba, pineapple, Noni fruit, curcuma, orange, apple, green plum, Indian gooseberry and any combinations thereof, and a total peptide content in the solid vegetable and fruit enzyme product is 437.92 mg/100 g.

9. The process of claim 5, wherein the solid vegetable and fruit enzyme product is pastille, powder or capsule.
